SEQUENCE LISTING (18 sequences)

SEQ ID NO: 1 - DNA, 2091 bp, Synechocystis sp.
atggacagtggccatggcgctcaatccaggataaagcttggtcagactgggtataaactgtcaacatatttctgcaagagtgggcccaattgggaaaatcaacctcaaatccattggaatagccttttttcaaccgtaaaaatccaactttctctcttcccttcttccttccatctgattatggttacgccaattaactaccattccatccattgcctggcggatatctgggctatcaccggagaaaattttgccgatattgtggccctcaacgatcgccatagtcatccccccgtaactttaacctatgcccaattgcgggaagaaattacagcttttgccgctggcctacagagtttaggagttaccccccatcaacacctggccattttcgccgacaacagcccccggtggtttatcgccgatcaaggcagtatgttggctggagccgtcaacgccgtccgttctgcccaagcagagcgccaggaattactctacatcctagaagacagcaacagccgtactttaatcgcagaaaatcggcaaaccctaagcaaattggccctagatggcgaaaccattgacctgaaactaatcatcctcctcaccgatgaagaagtggcagaggacagcgccattccccaatataactttgcccaggtcatggccctaggggccggcaaaatccccactcccgttccccgccaggaagaagatttagccaccctgatctacacctccggcaccacaggacaacccaaaggggtgatgctcagccacggtaatttattgcaccaagtacgggaattggattcggtgattattccccgccccggcgatcaggtgttgagcattttgccctgttggcactccctagaaagaagcgccgaatattttcttctttcccggggctgcacgatgaactacaccagcattcgccatttcaagggggatgtgaaggacattaaaccccatcacattgtcggtgtgccccggctgtgggaatccctctacgaaggggtacaaaaaacgttccgggaaaagtcccctgggcaacaaaagctaattaatttctttttcggcatttcccaaaaatatattttggccaaacgcattgccaataacctgagcttgaaccatctccacgcttcggcgatcgccaggttggtggcccggtgccaagccttggtgcttagtcctctccattacctcggggacaaaattgtctaccataaggtacgccaggccgctgggggcagactggaaactctcatttccggaggaggggcgttagctagacatttagatgatttttacgaaatcaccagcattcccgtcctggtgggctatggcttaacggaaacggccccagtaactaatgccagggtgcataaacataatttgcgctattcctctggccgccccattcctttcacagaaattcgtattgttgacatggaaaccaaggaggatttgccccccgaaacccaaggtcttgtgctaatccgtggtccccaggtgatgcagggctattacaacaagccggaagccaccgccaaagttttagaccaggaaggctggttcgacagcggtgacttaggctgggtaacgccccaaaatgatttgattctcaccggtcgggccaaggacaccattgtgctcagtaacggggaaaatgtggaaccccaacccattgaagatgcctgtttacgcagtgcctacattgaccagattatgctggtgggccaggatcaaaaatccttgggggctttgattgtgcccaacttcgatgcattgcaaaaatgggcagagacgaaaaatttacaaatcaccgtgccggaaccgtcggctagcagtgaagggatgcaggctagtggtttgtatgacccccaagtggtggggttaatgcggtcggagttgcatcgggaagtgcgcgatcgccctggctaccgagccgatgaccagattaaggatttccgttttatcccagcaccattttccctggaaaacggcatgatgacccaaaccttgaagctcaaacgaccagtggtaacccaaacttatcaacatttaattgacgaaatgttttaa

SEQ ID NO: 2 - DNA, 501 bp, artificial
  upstream fragment of slr1609 gene
gggcatccaccaacgctttggtgatgaacactggggaaaccccagaaatgaggggaggtaagggataggttgcccctgccgtagttcccttgattaaaaattccgcatcggcgatcgccgtcaattttcgatcagcgggggttttacccgccgcagaaatgcccggaattaaaccagtttccgtaaagcccaacacacagacaaacaccggtggacagtggccatggcgctcaatccaggataaagcttggtcagactgggtataaactgtcaacatatttctgcaagagtgggcccaattgggaaaatcaacctcaaatccattggaatagccttttttcaaccgtaaaaatccaactttctctcttcccttcttccttccatctgattatggttacgccaattaactaccattccatccattgcctggcggatatctgggctatcaccggagaaaattttgccgatattgtggccctcaacgatcgccatagtcatccc

SEQ ID NO: 3 - DNA, 501 bp, artificial
  downstream fragment of slr1609 gene
gcctacattgaccagattatgctggtgggccaggatcaaaaatccttgggggctttgattgtgcccaacttcgatgcattgcaaaaatgggcagagacgaaaaatttacaaatcaccgtgccggaaccgtcggctagcagtgaagggatgcaggctagtggtttgtatgacccccaagtggtggggttaatgcggtcggagttgcatcgggaagtgcgcgatcgccctggctaccgagccgatgaccagattaaggatttccgttttatcccagcaccattttccctggaaaacggcatgatgacccaaaccttgaagctcaaacgaccagtggtaacccaaacttatcaacatttaattgacgaaatgttttaagaacctgtttataaagtctgattctgccccctaaatcccccaatactgggggactttgacttagtttccccccaaacttggggggccaggggggcttttcaaacacgatctaaggcctatgggtaa

SEQ ID NO: 4 - DNA, 1145 bp, artificial
  kanamycin resistance gene ck2
ggatcctctagagtccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgactctagaggatcc

SEQ ID NO: 5 - DNA, 3058 bp, artificial
  kanamycin resistance gene ck2 and sucrose screening gene
gatatcggcattttcttttgcgtttttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacctccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagctagagtcgacgtcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggactctaga

SEQ ID NO: 6 - DNA, 1455 bp, artificial
  upstream fragment of psbA2 gene
cacatagttctgccagttgaggttgacgtaaccaaaggcaatttctaaaaattccttcacttcgtgggtttcccccgtggccaccacatagtcatcgggctgttcctgttgcaacatggcccacatggcccgtacatagtccttggcatagccccaatcccgcttggaatcgatattgcctaaatacaatttcttttgggtgccggccacaattctggcgatcgccctagtaattttcctggttacaaaggtttctccccggcggggggattcgtggttgaacaaaatgccgttacaggcgaataagtcataggattcccgatagttcaccgtttgccaatggccataaaccttggcacaggcgtagggactgcggggataaaagggggtggtttccttttggggaatctcctgcactttgccgaacatttccgaagaaccggcttgatagaaccttacttggatgccggtgcgatgttgataatcccgaatcgcttccaatagtcgtagcgtccccatggccactgaatctacagtgtattccggagaatcaaagctcacccgcacgtgggattgggcccccagattgtaaatctccgtcggtttgacatcttctaaaatgcggcgcagggtggtgccgtcggtcagatcaccataatgaagtcggagtttcgcctcaagatcatggggatcaacataaagatgatcaatgcggtcagtgttaaaggtagaagttcggcgaatgatgccatggacttggtagcccttttccaacaacaattcactcagataggagccatcttgccccgtgatgcctgtcagcaaaacaactttagactttgacattagttaatttttccccattgccccaaaatacatccccctaaaaatatcagaatccttgcccagatgcaggccttctggcgatcgccatggtgagcaacgattgcggctttagcgttccagtggatatttgctgggggttaatgaaacattgtggcggaacccagggacaatgtgaccaaaaaattcagggatatcaataagtattaggtatatggatcataattgtatgcccgactattgcttaaactgactgaccactgaccttaagagtaatggcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaacatcgacaaatacataaggaattataaccaaatg

SEQ ID NO: 7 - DNA, 600 bp, artificial
  downstream fragment of psbA2 gene
ttccttggtgtaatgccaactgaataatctgcaaattgcactctccttcaatggggggtgctttttgcttgactgagtaatcttctgattgctgatcttgattgccatcgatcgccggggagtccggggcagttaccattagagagtctagagaattaatccatcttcgatagaggaattatgggggaagaacctgtgccggcggataaagcattaggcaagaaattcaagaaaaaaaatgcctcctggagcattgaagaaagcgaagctctgtaccgggttgaggcctggggggcaccttattttgccattaatgccgctggtaacataaccgtctctcccaacggcgatcggggcggttcgttagatttgttggaactggtggaagccctgcggcaaagaaagctcggcttacccctattaattcgtttttccgatattttggccgatcgcctagagcgattgaatagttgttttgccaaggcgatcgcccgttacaattaccccaacacctatcaggcggtttatccggtcaaatgtaaccagcaacgacatctggtggaagccctggttcgctttgggcaaacttcccagtgtgga

SEQ ID NO: 8 - DNA, 30 bp, artificial
  primer
tacatatggacagtggccatggcgctcaat

SEQ ID NO: 9 - DNA, 31 bp, artificial
  primer
ccctcgagaaacatttcgtcaattaaatgtt

SEQ ID NO: 10 - DNA, 25 bp, artificial
  primer
tttaaatggtgatgaacactgggga

SEQ ID NO: 11 - DNA, 24 bp, artificial
  primer
gggatgactatggcgatcgttgag

SEQ ID NO: 12 - DNA, 23 bp, artificial
  primer
tgtttacgcagtgcctacattga

SEQ ID NO: 13 - DNA, 23 bp, artificial
  primer
cccataggccttagatcgtgttt

SEQ ID NO: 14 - DNA, 23 bp, artificial
  primer
cacatagatctgccagttgaggt

SEQ ID NO: 15 - DNA, 31 bp, artificial
  primer
gggcatatggttataattccttatgtatttg

SEQ ID NO: 16 - DNA, 22 bp, artificial
  primer
ttccttggtgtaatgccaactg

SEQ ID NO: 17 - DNA, 20 bp, artificial
  primer
tccacactgggaagtttgcc

SEQ ID NO: 18 - DNA, 3806 bp, artificial
  erythromycin resistance gene
gatatcggcattttcttttgcgtttttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacctccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagctagagtcgacctgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgcgaattcatcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgatatc

(19) United States
(12) Patent Application Publication
(10) Pub. No.: US 2013/0059350 A1
(43) Pub. Date: Mar. 7, 2013

(54) CONSTRUCTS AND METHODS FOR INCREASING YIELD OF FATTY ALCOHOLS IN CYANOBACTERIA

(71) Applicants: Xuefeng LU, Qingdao (CN); Qianqian GAO, Qingdao (CN); Weihua WANG, Qingdao (CN); Hui ZHAO, Qingdao (CN)

(72) Inventors: Xuefeng LU, Qingdao (CN); Qianqian GAO, Qingdao (CN); Weihua WANG, Qingdao (CN); Hui ZHAO, Qingdao (CN)

(73) Assignee: SHELL OIL COMPANY, Houston, TX (US)

(21) Appl. No.: 13/593,701

(22) Filed: Aug. 24, 2012

(30) Foreign Application Priority Data
Aug. 26, 2011 (CN) 201110246569.2

(51) Int. Cl. C12P 7/64; C12N 1/13; C12P 5/02; C12N 15/74

(52) U.S. Cl. 435/134; 435/320.1; 435/257.2; 435/471; 435/167

(57) ABSTRACT

A construct containing a gene operably linked to a promoter having activity in cyanobacteria, and vector and cyanobacterium comprising such construct for increasing the yield of fatty alcohols in cyanobacterium is provided.

The present application claims the benefit of Chinese Patent Application No. 201110246569.2, filed Aug. 26, 2011, the entire disclosure of which is hereby incorporated by reference.

### FIELD OF THE INVENTION

The present invention relates to a novel construct and a method for producing one or more fatty alcohols in cyanobacteria using such construct. Specifically, the present invention relates to a construct for increasing the yield of fatty alcohols in cyanobacteria, a vector comprising such construct, a cyanobacterium comprising such construct or transformed by such vector, and a method for increasing the yield of fatty alcohols in cyanobacteria, wherein the cyanobacteria have been modified for producing fatty alcohols.

### BACKGROUND OF THE INVENTION

With the diminishing supply of crude mineral oil, use of renewable energy sources is becoming increasingly important for the production of fuels and chemicals. These fuels and chemicals from renewable energy sources are often referred to as biofuels, respectively biochemicals. Biofuels and/or biochemicals for which the production does not compete with food production are preferred.

An example of a biofuel and/or biochemical is bio-ethanol. Technical routes for the production of bio-ethanol have already been developed and industrial production of bio-ethanol at a large scale may be achieved. However, ethanol as a fuel has some drawbacks, including: (1) low energy density; (2) high volatility; (3) problems caused by its high solubility in water, such as the increased toxicity to microorganisms during fermentation, the high cost for the removal of water phase during distillation separation process and the corrosion of pipelines during transportation.

An ideal biofuel preferably has properties such as a high energy density, a low moisture absorption and a low volatility. An ideal biofuel is further preferably compatible with existing engines and transport facilities.

Recently, high quality fatty-acid biofuels such as long chain fatty alcohols and long chain biologic hydrocarbons are drawing more and more attention from academia and industry. Professor Jay D Keasling, a biologist of synthesis, has written a review concerning the status and prospective of such biofuels (see the article of Keasling, J. D., S. K. Lee, H. Chou, T. S. Ham, and T. S. Lee (2008) titled “Metabolic engineering of microorganisms for biofuels production: from bugs to synthetic biology to fuels” in Curr. Opin. Biotech. Vol. 19, pages 556-563).

The research results of Professor Jay D Keasling and his collaborators were reported in Nature: fatty-acid biofuels such as fatty alcohols and wax esters were successfully synthesized in E. coli by means of metabolic engineering (see the article of Keasling, J. D., E. J. Steen, Y. S. Kang, G. Bokinsky, Z. H. Hu, A. Schirmer, A. McClure, and S. B. del Cardayre (2010) titled “Microbial production of fatty-acid-derived fuels and chemicals from plant biomass” in Nature pages 463-559: U182) Keasling, et al, 2010).

Further US biofuel company LS9 has worked on the production of biofuels in modified microorganisms such as E. coli and Saccharomyces cerevisiae by using genetic engineering methods (see WO 2009/140695 and WO 2009/140696).

At present, the microorganism systems used for studying biofuels are primarily heterotrophic microorganisms represented by E. coli and Saccharomyces cerevisiae.

In 2009 and 2010, several research groups described the production of biofuels using cyanobacteria. Angermayr et al. described cyanobacteria as a new generation of energy microorganisms (see the article of Angermayr, S. A., K. J. Hellingwerf, P. Lindblad, and M. J. T. de Mattos (2009) titled “Energy biotechnology with cyanobacteria” in Curr. Opin. Biotech. Vol. 20 pages 257-263). Professor F U Pengcheng from China University of Petroleum described the conversion from solar energy to bio-ethanol (yield of 5.2 mmol/OD₇₃₀/L/d) by co-expressing the genes of pyruvate decarboxylase and ethanol dehydrogenase derived from Zymomonas mobilis in Synechocystis sp. PCC6803 (see the article of Fu, P. C., and J. Dexter (2009) titled “Metabolic engineering of cyanobacteria for ethanol production” in Energ. Environ. Sci. Vol. 2 pages 857-864). The research group of Professor Anastasios Melis from University of California, Berkeley, described the production of isoprene in cyanobacteria (yield of 50 mg/g/d) by exogenously expressing the isoprene synthase gene of Pueraria montana in Synechocystis sp. PCC6803 (see the article of Melis, A., P. Lindberg, and S. Park. (2010), titled “Engineering a platform for photosynthetic isoprene production in cyanobacteria, using Synechocystis as the model organism” in Metabolic Engineering. Vol. 12 pages 70-79).

Professor James C. Liao from University of California, Los Angeles, further reported the effective production of isobutyraldehyde in Synechococcus elongatus PCC 7942 (highest yield of 6,230 μg/L/h) by means of genetic engineering (see the article of Cai, Y. P., and C. P. Wolk. (1990) titled “Use of a Conditionally Lethal Gene in Anabaena Sp-Strain Pcc-7120 to Select for Double Recombinants and to Entrap Insertion Sequences” in the Journal of Bacteriology Vol. 172 pages 3138-3145).

Curtiss et al described the production and secretion of free fatty acids in Synechocystis sp. PCC6803 (see the article of Curtiss, R., X. Y. Liu, and J. Sheng (2011), titled “Fatty acid production in genetically modified cyanobacteria” in P. Natl. Acad. Sci. (PNAS) USA. Vol. 108. pages 6899-6904).

### SUMMARY OF THE INVENTION

It has been found that the yield of fatty alcohols in cyanobacteria capable of producing fatty alcohols can be increased by increasing the expression level of fatty acyl-CoA synthetase.

Accordingly, in one aspect, the present invention provides a construct, comprising a gene operably linked to a promoter having activity in cyanobacteria, which gene is selected from the group consisting of:

1) fatty acyl-CoA synthetase genes;
2) genes of which the nucleotide sequences have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having fatty acyl-CoA synthetase activity; and
3) genes of which the nucleotide sequences are capable of hybridizing with the sequences of the genes listed in 1) (i.e. fatty acyl-CoA synthetase genes) under stringent hybridizing conditions, preferably highly stringent hybridizing conditions, and which encode a protein having fatty acyl-CoA synthetase activity.

The promoter having activity in cyanobacteria is preferably a constructive promoter or an inductive promoter. More preferably the promoter is selected from the group consisting of psbA2 promoter, rbc promoter, petE promoter, cmp promoter, sbt promoter or trc promoter. Most preferably the promoter has the sequence as shown in SEQ ID NO: 6.

In this aspect of the invention the gene is preferably a fatty acyl-CoA synthetase gene, more preferably a gene selected from the group consisting of slr1609 gene from Synechocystis sp. PCC6803; cce_{—}1133 from Cyanothece sp. ATCC 51142; SYNPCC7002_A0675 from Synechococcus 7002; syc0624_c from Synechococcus PCC 6301; Synpcc7942_{—}0918 from Synechococcus PCC 7942; and alr3602 from Anabaena PCC 7120. More preferably the gene has the sequence as shown in SEQ ID NO: 1.
In the first aspect of the invention the cyanobacteria are preferably cyanobacteria capable of producing fatty alcohols, which are modified by genetic engineering to express fatty acyl-CoA reductase; more preferably cyanobacteria capable of producing fatty alcohols which are selected from the group consisting of: Synechocystis sp. Syn-XT14, Syn-XT34 and Syn-XT51.

In another aspect the invention provides a vector, which comprises the above construct.

In yet another aspect the invention provides a cyanobacterium comprising the above construct and/or the above vector.

In another aspect the invention provides a kit, which comprises two constructs, wherein the first construct is the above construct and the second construct comprises a gene operably linked to a promoter having activity in cyanobacteria, which gene is selected from the group consisting of:

1) fatty acyl-CoA reductase genes;
2) genes of which the nucleotide sequences have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having fatty acyl-CoA reducase activity; and
3) genes of which the nucleotide sequences are capable of hybridizing with the sequences of the genes listed in 1) under stringent hybridizing conditions, preferably highly stringent hybridizing conditions, and which encode a protein having fatty acyl-CoA reducase activity.
Preferably, the promoter as comprised in the second construct is a constructive promoter or an inductive promoter, preferably psbA2 promoter, rbc promoter, petE promoter, cmp promoter, sbt promoter or trc promoter. Preferably, the fatty acyl-CoA reducase gene is selected from the group consisting of: far gene from Simmondsia chinensis; at3g11980 gene from Arabidopsis thaliana; far1 gene from mouse; far1 gene with optimized codon from mouse; far2 gene from mouse; at3g56700 gene from Arabidopsis thaliana; Francci3_{—}2276 from Frankia sp. CcI3; KRH_{—}18580 from Kocuria rhizophila DC2201; A20C1_{—}04336 from Actinobacterium PHSC20C1; HCH_{—}05075 from Hahella chejuensis KCTC 2396; Maqu_{—}2220 from Marinobacter aquaeolei VT8; and RED65_{—}09889 from Oceanobacter sp. RED65. Preferably, the second construct can further comprise a marker gene for screening the transformants of cyanobacteria, preferably kanamycin resistance gene, erythromycin resistance gene and spectinomycin resistance gene. And preferably the marker gene of the second construct is different from the marker gene of the first construct.

In yet another aspect the present invention provides a kit, comprising two vectors, wherein the first vector comprises the first construct as defined above, and the second vector comprises the second construct as defined above.

In further aspect the present invention provides a cyanobacterium, which comprises the first construct as defined above and/or the first vector as defined above, and comprises the second construct as defined above and/or the second vector as defined above. Preferably this cyanobacterium is the cyanobacterium GQ5 as deposited at the China General Microbiological Culture Collection Center (CGMCC), having its address at the Institute of Microbiology, Chinese Academy of Sciences, No. 1 West Beichen Road, Chaoyang District, Beijing 100 101, China, under Accession Number of CGMCC 4890 on May 20, 2011.

In another aspect the present invention provides a method for increasing the yield of fatty alcohols in one or more cyanobacteria capable of producing fatty alcohols, comprising introducing any of the above constructs and/or any of the above vectors into such one or more cyanobacteria. Preferably the one or more cyanobacteria capable of producing fatty alcohols are one or more cyanobacteria that are modified by genetic engineering to express fatty acyl-CoA reducase. preferably the one or more cyanobacteria capable of producing fatty alcohols are selected from the group consisting of Synechocystis sp., Syn-XT14, Syn-XT34 and Syn-XT51. And preferably the construct is integrated into the genome of the one or more cyanobacteria.

In an yet another aspect the present invention provides a method for producing a fatty alcohol in one or more cyanobacteria, the method comprising:

1) introducing the first construct as defined above and/or the first vector as defined above, as well as the second construct as defined above and/or the second vector as defined above, into a cyanobacterium; and
2) culturing the cyanobacterium obtained in step 1), and obtaining fatty alcohols from the culture.
Preferably the cyanobacterium is Synechocystis sp. PCC6803. Preferably the first construct and/or the second construct is integrated into the genome of the cyanobacterium. More preferably, the cyanobacterium obtained in step 1) is cyanobacterium GQ5 as deposited in China General Microbiological Culture Collection Center (CGMCC) under Accession Number of CGMCC 4890 on May 20, 2011. Conveniently the obtained fatty alcohols may be converted further to obtain alkanes, and optionally the obtained alkanes may be blended with one or more additives into a fuel and/or a chemical.

The construct(s) and the kit can advantageously be used for increasing the yield of fatty alcohols in cyanobacteria capable of producing fatty alcohols.

### BRIEF DESCRIPTION OF THE DRAWINGS

The invention is further illustrated by the following non-limiting figures:

FIG. 1 shows the basic structure of plasmid pGQ7. The plasmid pGQ7 is obtained by cloning slr1609 gene (SEQ ID NO: 1) from Synechocystis sp. PCC6803 into plasmid pET21b (Novagen) using restriction enzymes NdeI and XhoI.

FIG. 2 shows coupling reactions as also described in the article of Hosaka, K., M. Mishina, T. Tanaka, T. Kamiryo, and S. Numa (1979) titled “Acyl-Coenzyme-a Synthetase-I from Candida-Lipolytica—Purification Properties and Immunochemical Studies” published in Eur J Biochem Vol. 93 pages 197-203.

FIG. 3 shows the basic structure of plasmid pXT68. The plasmid pXT68 is obtained by cloning the upstream fragment (SEQ ID NO: 6, comprising psbA2 promoter) and downstream fragment (SEQ ID NO: 7) of psbA2 gene from Synechocystis sp. PCC6803 and kanamycin resistance gene ck2 (SEQ ID NO: 4) into plasmid pMD18-T (Takara, Catalog No.: D101A).

FIG. 4 shows the basic structure of plasmid pGQ49. The plasmid pGQ49 is obtained by cloning slr1609 gene (SEQ ID NO: 1) into plasmid pXT68 using restriction enzymes NdeI and XhoI. In the plasmid, slr1609 gene is operably linked to psbA2 promoter, so that the expression thereof is driven by the psbA2 promoter.

FIG. 5 shows the basic structure of plasmid pGQ17. The plasmid pGQ17 is obtained by cloning the upstream fragment (SEQ ID NO: 2) and downstream fragment (SEQ ID NO: 3) of the slr1609 gene and the kanamycin resistance gene ck2 (SEQ ID NO: 4) into the plasmid pMD18-T.

FIG. 6 shows the production of fatty alcohols in the cells of Synechocystis sp. Syn-XT14 after 10 days of culturing, as determined by gas chromatography coupled with mass spectrometry, wherein C15-OH represents 1-pentadecanol (used as internal standard), C16-OH represents 1-hexadecanol, C18-OH represents 1-octadecanol, the vertical axis represents abundance, and the horizontal axis represents time (unit: minute).

FIG. 7 shows the production of fatty alcohols in the cells of Synechocystis sp. GQ6 after 10 days of culturing, as determined by gas chromatography coupled with mass spectrometry, wherein C15-OH represents 1-pentadecanol (used as internal standard), C16-OH represents 1-hexadecanol, C18-OH represents 1-octadecanol, the vertical axis represents abundance, and the horizontal axis represents time (unit: minute).

FIG. 8 shows the production of fatty alcohols in the cells of Synechocystis sp. GQ5 after 10 days of culturing, as determined by gas chromatography coupled with mass spectrometry, wherein C15-OH represents 1-pentadecanol (used as internal standard), C16-OH represents 1-hexadecanol, C18-OH represents 1-octadecanol, the vertical axis represents abundance, and the horizontal axis represents time (unit: minute).

### DESCRIPTION OF THE SEQUENCES

Sequence listing submitted in computer readable form submitted electronically herewith is hereby interpreted by reference.

SEQ ID NO: 1: the nucleotide sequence of slr1609 gene (NCBI ID: NC_{—}000911.1) from Synechocystis sp. PCC6803. SEQ ID NO: 2: the nucleotide sequence of the upstream fragment of slr1609 gene, which is obtained by amplifying the genome DNA of Synechocystis sp. PCC6803 with primers 1609 kuF (SEQ ID NO: 10) and 1609 kuR (SEQ ID NO: 11).

SEQ ID NO: 3: the nucleotide sequence of the downstream fragment of slr1609 gene, which is obtained by amplifying the genome DNA of Synechocystis sp. PCC6803 with primers 1609 kdF (SEQ ID NO: 12) and 1609 kdR (SEQ ID NO: 13).

SEQ ID NO: 4: the nucleotide sequence of the kanamycin resistance gene ck2 (NCBI ID: NC_{—}003239.1) on plasmid pRL446 (see the article of Elhai, J., and C. P. Wolk (1988) titled “A Versatile Class of Positive-Selection Vectors Based on the Nonviability of Palindrome-Containing Plasmids That Allows Cloning into Long Polylinkers” Gene Vol. 68 pages 119-138).

SEQ ID NO: 5: the nucleotide sequence of the kanamycin resistance gene ck2 (NCBI ID NC_{—}003239.1) and sucrose screening gene (NCBI IDNC_{—}000964.3) on plasmid pRL446.

SEQ ID NO: 6: the nucleotide sequence of the upstream fragment of psbA2 gene, which is obtained by amplifying the genome DNA of Synechocystis sp. PCC6803 with primers Pd1-2-f (SEQ ID NO: 14) and Pd1-2-r (SEQ ID NO: 15).

SEQ ID NO: 7: the nucleotide sequence of the downstream fragment of psbA2 gene, which is obtained by amplifying the genome DNA of Synechocystis sp. PCC6803 with primers pD1-2d-1 (SEQ ID NO: 16) and pD1-2d-2 (SEQ ID NO: 17).

SEQ ID NO: 8: the nucleotide sequence of primer 1609NdeI.

SEQ ID NO: 9: the nucleotide sequence of primer 1609R.

SEQ ID NO: 10: the nucleotide sequence of primer 1609 kuF.

SEQ ID NO: 11: the nucleotide sequence of primer 1609 kuR.

SEQ ID NO: 12: the nucleotide sequence of primer 1609 kdF.

SEQ ID NO: 13: the nucleotide sequence of primer 1609 kdR.

SEQ ID NO: 14: the nucleotide sequence of primer Pd1-2-f.

SEQ ID NO: 15: the nucleotide sequence of primer Pd1-2-r.

SEQ ID NO: 16: the nucleotide sequence of primer pD1-2d-1.

SEQ ID NO: 17: the nucleotide sequence of primer pD1-2d-2.

SEQ ID NO: 18: the nucleotide sequence of erythromycin resistance gene (NCBI ID: NC_{—}015291.1) on plasmid pRL271 (see also the article of Elhai, J., and C. P. Wolk (1988) titled “A Versatile Class of Positive-Selection Vectors Based on the Nonviability of Palindrome-Containing Plasmids That Allows Cloning into Long Polylinkers” published in Gene Vol. 68 pages 119-138).

### DETAILED DESCRIPTION OF THE INVENTION

The inventors of the present invention have already successfully produced fatty alcohols in cyanobacteria by expressing exogenous fatty acyl-CoA reductase in Synechocystis sp. PCC6803 (see WO2011086189, the content of which is incorporated herein by reference in its entirety). It would, however, be an advancement in the art to further increase the yield of fatty alcohols in cyanobacteria. This may help to promote the application of cyanobacteria for the synthesis of fatty alcohols as a biofuel and may help the sustainable development of the economy and society.

In the present application, the inventors established a route for synthesizing fatty alcohols in the cells of cyanobacteria, wherein the energy for synthesizing the fatty alcohols can advantageously be solar energy, and the carbon source can advantageously be carbon dioxide. This route may allow one to utilize solar energy for fixing carbon dioxide and synthesizing fatty alcohols in cells of a photosynthetic microorganism such as a cyanobacteria.

One of the advantages of the present invention is that fatty alcohols may be synthesized by using solar energy to fix carbon dioxide in the photosynthetic microorganism cyanobacteria, wherein the energy for synthesizing fatty alcohols is solar energy and the carbon source is carbon dioxide. Thus, the production of biofuels utilizing this technology would not be restricted by the lack of raw materials, and the use of such biofuels would not increase carbon emission, i.e., such biofuels are real zero emission biofuels.

Further the present invention may allow one to advantageously increase the yield of fatty acyl-CoA in cyanobacteria by increasing the level of expression of fatty acyl-CoA synthetase in cyanobacteria. This in turn may advantageously allow one to increase the yield of downstream product fatty alcohols.

Hence, another advantage of the present invention may lie in the increase of the yield of fatty alcohols in cyanobacteria. This can assist in providing beneficial conditions for producing biofuel fatty alcohols at a large scale by using cyanobacteria.

Without wishing to be limited by any kind of theory, the inventors believe that the mechanism for producing fatty alcohols in cyanobacteria may be as follows: in cyanobacteria, free fatty acids can be activated by fatty acyl-CoA synthetase to form fatty acyl-CoAs, and the fatty acyl-CoAs can be further converted into fatty alcohols under the catalysis of fatty acyl-CoA reducase. Wild-type cyanobacteria (e.g., Synechocystis sp. PCC6803) can naturally express fatty acyl-CoA synthetase (its coding gene being slr1609 gene, see: for example, NCBI ID: NC_{—}000911.1), but do not express fatty acyl-CoA reducase. The inventors have successfully constructed a route for synthesis of fatty alcohols in cells of cyanobacteria by allowing cyanobacteria to express fatty acyl-CoA reducase (e.g., by genetic engineering method), thereby achieving the synthesis of fatty alcohols in cyanobacteria. The inventors further believe that the level of expression of endogenous fatty acyl-CoA synthetase in cyanobacteria may be relatively low, and may not meet the requirements for production of fatty alcohols in large scale. Without wishing to be limited by any kind of theory, the inventors believe that the yield of fatty acyl-CoA can be elevated by increasing the level of expression of fatty acyl-CoA synthetase in cyanobacteria (e.g., by high expression of endogenous fatty-CoA synthetase, or by exogenous expression of fatty-CoA synthetase), thereby increasing the yield of fatty alcohols as downstream product.

### DEFINITION OF TERMS

In the present invention, unless indicated otherwise, all scientific and technological terms used have the meaning associated with these terms as known by one skilled in the art. In addition, the laboratory procedures of cell culture, molecular genetics, nucleic acid chemistry, organic chemistry are all conventional procedures well known by one skilled in the corresponding fields. Unless indicated otherwise, the molecular biological experimental methods used in the present invention are carried out substantially in accordance with the methods as described by Sambrook J et al., Molecular Cloning: A Laboratory Manual (Second Edition), Cold Spring Harbor Laboratory Press, 1989, and F. M. Ausubel et al., Short Protocols in Molecular Biology, John Wiley & Sons, Inc., 1995, or according to the instructions of products.

In addition, the following definitions and explanations of related terms are provided for better understanding of the present invention:

By a “construct” is herein understood a segment comprising one or more nucleic acids, for example a DNA fragment. The construct is suitably an artificially constructed segment of one or more nucleic acids. The construct can be used to subclone one or more of the nucleic acids, for example a DNA fragment, into a vector.

By a “Cyanobacterium” is herein understood a member from the group of photoautotrophic prokaryotic microorganisms, which can utilize solar energy and fix carbon dioxide. Cyanobacteria are sometimes also referred to as blue-green algae. In the present invention, the terms “cyanobacteria” and “blue-green algae” are used interchangeably. A representative of unicellular cyanobacteria is Synechocystis sp. PCC6803.

As used in the present invention, “cyanobacteria capable of producing fatty alcohols” refer to cyanobacteria that are able to express fatty acyl-CoA reductase, thereby being capable of producing fatty alcohols. Preferably the cyanobacteria have been modified by gene engineering to be able to express fatty acyl-CoA reductase. The cyanobacteria can be modified by using the methods well known in the art so that they can express fatty acyl-CoA reductase, for example, by introducing a gene coding for fatty acyl-CoA reductase into the cyanobacteria, or integrating said gene into the genome of cyanobacteria. Examples of cyanobacteria capable of producing fatty alcohols include but are not limited to Synechocystis sp. Syn-XT14, Syn-XT34 and Syn-XT51 such as for example described in above mentioned WO2011086189.

As used in the present invention, “fatty acyl-CoA synthetase” is an enzyme capable of catalyzing the reaction of free fatty acid with ATP and CoA to produce fatty acyl-CoA. Fatty acyl-CoA synthetase is herein also referred to as fatty acyl-Coenzyme A synthetase. The genes encoding fatty acyl-CoA synthetase are well known in the art, including but not being limited to: slr1609 gene from Synechocystis sp. PCC6803 (e.g., see: NCBI ID: NC_{—}000911.1); cce_{—}1133 from Cyanothece sp. ATCC 51142 (e.g., see: NCBI ID: NC_{—}010546.1); SYNPCC7002_A0675 from Synechococcus 7002 (e.g., see: NCBI ID: NC_{—}010475.1); syc0624_c from Synechococcus PCC 6301 (e.g., see: NCBI ID: NC_{—}006576.1); Synpcc7942_{—}0918 from Synechococcus PCC 7942 (e.g., see: NCBI ID: NC_{—}007604.1); and alr3602 from Anabaena PCC 7120 (e.g., see: NCBI ID: NC_{—}003272.1).

By a “Fatty acyl-CoA reductase” (Far) is understood an enzyme capable of catalyzing the conversion reaction of fatty acyl-CoA to fatty alcohols. Fatty acyl-CoA reductase is herein also referred to as fatty acyl-Coenzyme A reductase. Genes for encoding fatty acyl-CoA reductase are well known in the art, including but not being limited to: far gene from Simmondsia chinensis (e.g., see: WO2011086189 herein incorporated by reference); at3g11980 gene from Arabidopsis thaliana (e.g., see WO2011086189 herein incorporated by reference); far1 gene from mouse (e.g., see: NCBI ID: BC007178); far1 gene with optimized codon from mouse; far2 gene from mouse (e.g., see: NCBI ID: BC055759); or at3g56700 gene from Arabidopsis thaliana (e.g., see: NCBI ID: NC_{—}003074.8). Other suitable fatty acyl-CoA reductase genes include, for example: Francci3_{—}2276 from Frankia sp. CcI3 (e.g., see: NC_{—}007777); KRH_{—}18580 from Kocuria rhizophila DC2201 (e.g., see; NC_{—}010617); A20C1_{—}04336 from Actinobacterium PHSC20C1 (e.g., see: NZ_AAOB01000003); HCH_{—}05075 from Hahella chejuensis KCTC 2396 (e.g., see: NC_{—}007645); Maqu_{—}2220 from Marinobacter aquaeolei VT8 (e.g., see: NC_{—}008740); and RED65_{—}09889 from Oceanobacter sp. RED65 (e.g., see: NZ_AAQH01000001).

As used in the present invention, “vector” refers to a nucleic acid vehicle capable of being inserted with a DNA fragment (e.g., a desired gene) to allow the DNA fragment (e.g., the desired gene) to be transferred into one or more recipient cells. The recipient cell is sometimes also referred to as host cell. When the vector allows the inserted DNA fragment to be expressed, the vector is also known as an expression vector. A vector can be introduced into a host cell by transformation, transduction or transfection to express the carried DNA fragment in the host cell. Suitable vectors are well known by those skilled in the art and include but are not limited to plasmids, phages, coemids, etc.

As used in the present invention, a DNA fragment (e.g., a gene of interest) can be operably linked to an expression control sequence to carry out the constitutive or inductive expression of the DNA fragment (e.g., the gene of interest). As used in the present invention, “operable linked to” means that a molecule is linked in a way that the expected function can be achieved. For example, a gene encoding sequence can be operably linked to an expression control sequence so that the expression control sequence can regulate the expression of the gene encoding sequence. As used in the present invention, “expression control sequence” is a control sequence that may be required for the expression of a gene, which is well known in the art. An expression control sequence preferably comprises a promoter, a transcription terminator, and/or potentially other sequences such as an enhancer sequence.

The construct of the present invention comprises a gene operably linked to a promoter having activity in cyanobacteria. As used in the present invention a promoter is preferably understood to be a regulatory region of DNA located upstream of a gene, providing a control point for regulated gene transcription. Examples of such promotors include but are not limited to a rbc promoter, a petE promoter and/or a psbA2 promoter such as described below. In a preferred embodiment the promotor is chosen from the group consisting of a rbc promoter, a petE promoter, a psbA2 promoter or a combination thereof.

As used in the present invention, “rbc promoter” (also abbreviated herein as “Prbc”) refers to the promoter of the operon encoding ribulose-1,5-bisphosphate carboxylase/oxygenase (Rubisco) catalyzing the first reaction of Calvin cycle of the photosynthesis in Synechocystis sp. PCC6803 genome (see also WO2011086189). Prbc is active in cyanobacteria, and its sequence has been disclosed in WO2011086189 herein incorporated by reference.

As used in the present invention, “petE promoter” (also abbreviated herein as “PpetE”) refers to the promoter of gene petE encoding plastocyanin (PC) (see also WO2011086189). Plastocyanin is an electron carrier for transferring electron from cytochrome b6/f complex to photosystem I in photosynthesis. PpetE is active in cyanobacteria, and its sequence has been disclosed in WO2011086189 herein incorporated by reference.

As used in the present invention, “psbA2 promoter” (also abbreviated herein as “PpsbA2”) refers to a promoter of gene psbA2 encoding Photosystem II D1 protein. Photosystem II D1 protein is an important component of photosystem II, which is in charge of electron transfer. PpsbA2 is active in cyanobacteria, and can have the sequence as shown in SEQ ID NO: 6, for example. Previous studies describe that the deletion of psbA2 gene does not influence the physiological activities of Synechocystis sp. PCC6803 (i.e., the site of the gene is a neutral site of Synechocystis sp. PCC6803 genome) (see the article of Salih, G. F., and C. Jansson (1997), titled “Activation of the silent psbA1 gene in the cyanobacterium Synechocystis sp strain 6803 produces a novel and functional D1 protein” published in Plant Cell. Vol 9 pages 869-878).

In one preferable embodiment of the present invention, a 1.5 kb upstream fragment (for example SEQ ID NO: 6) comprising psbA2 promoter of psbA2 gene and the 600 bp downstream fragment (for example SEQ ID NO: 7) are cloned, respectively, for integrating psbA2 promoter and fatty acyl-CoA synthetase gene (e.g., fatty acyl-CoA synthetase gene slr1609 of Synechocystis sp. PCC6803) to the psbA2 gene site by homologous recombination, so as to over-express fatty acyl-CoA synthetase in cyanobacteria.

The construct of the present invention can comprise genes of which the nucleotide sequences are capable of hybridizing with the sequences of fatty acyl-CoA synthetase genes under stringent hybridizing conditions and which encode a protein having fatty acyl-CoA synthetase activity.

As used in the present invention, the term “hybridization” or “hybridizing” is intended to mean the process during which, under suitable conditions, two nucleic acid sequences bond to one another with stable and specific hydrogen bonds so as to form a double strand. These hydrogen bonds can form between the complementary bases adenine (A) and thymine (T) or uracil (U), which may then be referred to as an A-T bond; or between the complementary bases guanine (G) and cytosine (C), which may then be referred to as a G-C bond. The hybridization of two nucleic acid sequences may be total (reference is then made to complementary sequences), i.e. the double strand obtained during this hybridization comprises only A-T bonds and C-G bonds. Or the hybridization may be partial (reference is then made to sufficiently complementary sequences), i.e. the double strand obtained comprises A-T bonds and C-G bonds allowing the double strand to form, but also bases not bonded to a complementary base.

The hybridization between two complementary sequences or sufficiently complementary sequences depends on the operating conditions that are used, and in particular the stringency. The stringency may be understood to denote the degree of homology; the higher the stringency, the higher percent homology between the sequences. The stringency may be defined in particular by the base composition of the two nucleic sequences, and/or by the degree of mismatching between these two nucleic sequences. By varying the conditions (for example salt concentration and temperature), a given nucleic acid sequence may be allowed to hybridize only with its exact complement (high stringency) or with any somewhat related sequences (relaxed or low stringency). Increasing the temperature or decreasing the salt concentration may tend to increase the selectivity of a hybridization reaction.

As used in the present invention the phrase “hybridizing under stringent hybridizing conditions” is preferably understood to refer to hybridizing under conditions of a certain stringency.

In a preferred embodiment the “stingent hybridizing conditions” are conditions where homology of the two nucleic acid sequences is at least 70%, more preferably at least 80%, still more preferably at least 90% complete, that is, conditions where hybridization is only possible if the double strand obtained during this hybridization comprises respectively preferably at least 70%, more preferably at least 80%, still more preferably at least 90% of A-T bonds and C-G bonds.

In a more preferred embodiment the “stringent hybridizing conditions” are “highly stringent hybridizing conditions”. Preferably the “highly stringent hybridizing conditions” are conditions where the homology of the two nucleic acid sequences is at least 95%, more preferably at least 98%, still more preferably at least 99% and most preferably 100% complete, that is, conditions where hybridization is only possible if the double strand obtained during this hybridization comprises respectively preferably at least 95%, more preferably at least 98%, still more preferably at least 99% and most preferably 100% of A-T bonds, A and C-G bonds.

Most preferably “highly stringent hybridizing conditions” are conditions where a double strand can only be obtained if such a double strand comprises only A-T bonds and C-G bonds.

As indicated above, the stringency may depend on the reaction parameters, such as the concentration and the type of ionic species present in the hybridization solution, the nature and the concentration of denaturing agents and/or the hybridization temperature. The appropriate conditions can be determined by those skilled in the art.

As is known in the art, conditions for hybridizing nucleic acid sequences to each other can be described as ranging from low to high stringency. Reference herein to hybridization conditions of low stringency are preferably understood to refer to conditions including from at least about 0% to at most about 15% v/v formamide and from at least about 1 M to at most about 2 M salt for hybridization, and from at least about 1 M to at most about 2 M salt for washing conditions. Preferably, the temperature for hybridization conditions of low stringency is from about 25° C., more preferably from about 30° C. to about 42° C. Reference herein to hybridization conditions of medium stringency are preferably understood to refer to conditions including from at least about 16% v/v to at most about 30% v/v formamide and from at least about 0.5 M to at most about 0.9 M salt for hybridization, and from at least about 0.5 M to at most about 0.9 M salt for washing conditions. Reference herein to hybridization conditions of high stringency are preferably understood to refer to conditions including from at least about 31% v/v to at most about 50% v/v formamide and from at least about 0.01 M to at most about 0.15 M salt for hybridization, and from at least about 0.01 M to at most about 0.15 M salt for washing conditions. Preferably, washing is carried out at a temperature Tₘ=69.3+0.41 (G+C) % where Tₘ is in degrees Centigrade and (G+C) % refers to the mole percentage of guanine plus cytosine; in line with the article of J. Marmur et al. titled “Determination of the base composition of deoxyribonucleic acid from its thermal denaturation temperature”, published in Journal of Molecular Biology volume 5, issue 1, July 1962, pages 109-118 incorporated herein by reference. However, the Tₘ of a duplex DNA may decrease by 1° C. with every increase of 1% in the number of mismatch base pairs in line with the article of W. M. Bonner et al. titled “A Film Detection Method for Tritium-Labelled Proteins and Nucleic Acids in Polyacrylamide Gels”, published in the European Journal of Biochemistry, volume 46, issue 1, 1974, pages 83-88. Formamide is optional in these hybridization conditions. Accordingly, a particularly preferred non-limiting example of a hybridization condition of low stringency is 6×SSC (Standard Sodium Citrate) buffer, 1.0% w/v SDS (Sodium Dodecyl Sulfate) at a temperature in the range from 25° C. to 42° C.; a particularly preferred non-limiting example of a hybridization condition of medium stringency is 2×SSC (Standard Sodium Citrate) buffer, 1.0% w/v SDS (Sodium Dodecyl Sulfate) at a temperature in the range from 20° C. to 65° C.; and a particularly preferred non-limiting example of a hybridization conditions of high stringency is 0.1×SSC (Standard Sodium Citrate) buffer, 0.1% w/v SDS (Sodium Dodecyl Sulfate) at a temperature of at least 65° C. An extensive guide to the hybridization of nucleic acids can be found in Tijssen (1993) “Laboratory Techniques in Biochemistry and Molecular Biology—Hybridization with Nucleic Acid Probes”, Part I, Chapter 2 (Elsevier, New York); Ausubel et al., eds. (1995) “Current Protocols in Molecular Biology”, Chapter 2 (Greene Publishing and Wiley-Interscience, New York); and/or Sambrook et al. (1989) “Molecular Cloning: A Laboratory Manual” (2d ed., Cold Spring Harbor Laboratory Press, Plainview, N.Y.).

As used in the present invention, The term “identity” or “percent identity” refers to the sequence identity between two amino acid sequences or between two nucleic acid sequences. To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). For example, a “percent identity” is calculated by comparing two optimally aligned sequences over the window of comparison, determining the number of positions at which the identical nucleic acid base or the identical amino acid residue occurs in both sequences to yield the number of matched positions, dividing the number of matched positions by the total number of positions in the window of comparison (i.e., the window size), and multiplying the result by 100 to yield the percentage of sequence identity.

For example, if 6 out of 10 positions in two sequences are identical, these two sequences have a sequence identity of 60%.
Optimal alignment of sequences for comparison can be conducted, for example, by using a computer program such as Align program (DNAstar, Inc.) which is based on the method of Needleman, et al. (J. Mol. Biol. 48:443-453, 1970).
The percent identity between two nucleotide sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Percent identities involved in the embodiments of the present invention include at least about 60% or at least about 65% or at least about 70% or at least about 75% or at least about 80% or at least about 85% or at least about 90% or above, such as about 95% or about 96% or about 97% or about 98% or about 99%, such as at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The Cyanobacteria (also known as blue-green algae) in this invention preferably comprise a group of prokaryotic microorganisms capable of performing plant type oxygenic photosynthesis.

The use of cyanobacteria may have the following advantages: (1) cyanobacteria are capable of absorbing solar energy and fixing carbon dioxide as carbon source for autotrophic growth, thereby having low cost for culturing; (2) cyanobacteria are ancient microorganisms and have lived on the earth for billions of years, so that they have remarkable adaptability to the environments, and they grow quickly; (3) cyanobacteria are convenient for genetic manipulations, because their genetic background is clear and genomic sequencing of many species of cyanobacteria has been completed which facilitates the genetic engineering of cyanobacteria.

Examples of cyanobacterium include Synechococcus PCC 6301, Anabaena sp. strain PCC 7120, Synechococcus PCC 7002, Synechococcus elongatus sp. strain PCC 7942 and Synechocystis sp. PCC6803.

Synechocystis sp. PCC6803 is the most preferred cyanobacteria, because for Synechocystis sp. PCC6803 the whole genome sequencing had been completed in 1996. It has been described as one of the ideal models for the research of biofuel synthesis (see the article of Angermayr, S. A., K. J. Hellingwerf, P. Lindblad, and M. J. T. de Mattos (2009) titled “Energy biotechnology with cyanobacteria” in Curr. Opin. Biotech. Vol. 20 pages 257-263).

In one aspect, the present invention provides a construct, wherein the construct comprises a gene operably linked to a promoter having activity in cyanobacteria, which gene is selected from the group consisting of:

1) fatty acyl-CoA synthetase genes;
2) genes of which the nucleotide sequences have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having fatty acyl-CoA synthetase activity; and
3) genes of which the nucleotide sequences are capable of hybridizing with the sequences of the genes listed in 1) (i.e. fatty acyl-CoA synthetase genes) under stringent hybridizing conditions, preferably highly stringent hybridizing conditions, and which encode a protein having fatty acyl-CoA synthetase activity.

The embodiments of the present invention employ a promoter having activity in cyanobacteria. This promoter suitably drives the expression of fatty acyl-CoA synthetase and/or the expression of fatty acyl-CoA reductase in cyanobacteria. In this manner the characteristics of cyanobacteria as photosynthetic organism can be utilized to absorb solar energy, fix carbon dioxide and synthesize an increased amount of fatty alcohols as biofuels.

In the present invention, the promoter can be a constructive promoter or an inductive promoter. Examples of the promoter include but are not limited to, psbA2 promoter, rbc promoter, petE promoter, cmp promoter (as described in the article by Liu, X., S. Fallon, J. Sheng, and R. Curtiss, 3rd (2011) titled “CO2-limitation-inducible Green Recovery of fatty acids from cyanobacterial biomass” published in Proc Natl Acad Sci USA. Vol. 108 pages 6905-6908), sbt promoter (as described in the article by Liu, X., S. Fallon, J. Sheng, and R. Curtiss, 3rd (2011) titled “CO2-limitation-inducible Green Recovery of fatty acids from cyanobacterial biomass” published in Proc Natl Acad Sci USA. Vol. 108 pages 6905-6908) or trc promoter (as described in the article by Atsumi, S., W. Higashide, and J. C. Liao (2009) titled “Direct photosynthetic recycling of carbon dioxide to isobutyraldehyde” published in Nat Biotechnol. 27:1177-U1142).

In a preferred embodiment the promotor having activity in cyanobacteria is chosen from the group consisting of a rbc promoter, a petE promoter, a psbA2 promoter or a combination thereof.

In another preferred embodiment, the promoter has the sequence as shown in SEQ ID NO: 6.

In still another preferred embodiment, the construct comprises an upstream fragment and a downstream fragment of the psbA2 gene. The upstream fragment and the downstream fragment of the psbA2 gene are preferably located respectively at the two ends of the construct, so that the construct can be integrated at the site of the psbA2 gene in the genome of cyanobacteria by homologous recombination. Preferably such an upstream fragment of the psbA2 gene has the sequence as shown in SEQ ID NO: 6. Preferably such a downstream fragment of the psbA2 gene has the sequence as shown in SEQ ID NO: 7.

Examples of the fatty acyl-CoA synthetase gene include but are not limited to: slr1609 gene from Synechocystis sp. PCC6803 (see: e.g., NCBI ID: NC_{—}000911.1); cce_{—}1133 from Cyanothece sp. ATCC 51142 (e.g., see: NCBI ID: NC_{—}010546.1); SYNPCC7002_A0675 from Synechococcus 7002 (e.g., see: NCBI ID: NC_{—}010475.1); syc0624_c from Synechococcus PCC 6301 (e.g., see: NCBI ID: NC_{—}006576.1); Synpcc7942_{—}0918 from Synechococcus PCC 7942 (e.g., see: NCBI ID: NC_{—}007604.1); and alr3602 from Anabaena PCC 7120 (e.g., see: NCBI ID: NC_{—}003272.1). In another preferred embodiment, the gene has the sequence as shown in SEQ ID NO: 1.

In a preferred embodiment, the cyanobacteria capable of producing fatty alcohols are those that are modified by genetic engineering to express fatty acyl-CoA reducase and thus can produce fatty alcohols. For example, the cyanobacteria capable of producing fatty alcohols can be obtained by introducing a gene encoding fatty acyl-CoA reducase into cyanobacteria, or integrating the gene into the genome of cyanobacteria. Examples of cyanobacteria capable of producing fatty alcohols include but are not limited to Synechocystis sp. Syn-XT14, Syn-XT34 and Syn-XT51 such as described in WO2011086189, the content of which is incorporated herein by reference.

Further, the construct may comprise a marker gene for screening transformants of cyanobacteria. The marker gene can be located upstream or downstream of the promoter having activity in cyanobacteria. In a preferred embodiment the marker gene is located upstream of the promoter having activity in cyanobacteria.

Examples of such a marker gene include but are not limited to kanamycin resistance gene (NCBI ID: NC_{—}003239.1), erythromycin resistance gene (NCBI ID: NC_{—}015291.1) and spectinomycin resistance gene (such as described in WO2011086189, the content of which is incorporated herein by reference). In a preferred embodiment, the marker gene is a kanamycin resistance gene having for example the sequence as shown in SEQ ID NO: 4. In another preferred embodiment, the marker gene is the Omega fragment of the spectinomycin resistance gene, such as described in WO2011086189, the content of which is incorporated herein by reference) In a second aspect the invention provides a vector, which comprises the construct as described above.

Examples of vectors include but are not limited to cloning vectors and expression vectors. In a preferred embodiment, the vector is for example a plasmid, phage or coemid.

In a third aspect the invention provides a cyanobacterium comprising the above construct and/or the above vector and/or a cyanobacterium transformed with the above vector.

As indicated earlier, the cyanobacterium is preferably a cyanobacterium capable of producing fatty alcohols. In a preferred embodiment, the cyanobacterium is the cyanobacteria GQ5 as deposited in China General Microbiological Culture Collection Center (CGMCC) under Accession Number of CGMCC 4890 on May 20, 2011.

In a fourth aspect the invention provides a kit, which comprises two constructs, wherein the first construct is the above construct and the second construct comprises a gene operably linked to a promoter having activity in cyanobacteria, which gene is selected from the group consisting of:
1) fatty acyl-CoA reductase genes;
2) genes of which the nucleotide sequences have at least 80% identity, preferably at least 85% identity, more preferably at least 90% identity, more preferably at least 95% identity, such as at least 96% identity, at least 97% identity, at least 98% identity, or at least 99% identity, to the sequences of the genes listed in 1), and which encode a protein having fatty acyl-CoA reducase activity; and
3) genes of which the nucleotide sequences are capable of hybridizing with the sequences of the genes listed in 1) under stringent hybridizing conditions, preferably highly stringent hybridizing conditions, and which encode a protein having fatty acyl-CoA reducase activity.

Preferably, the promoter as comprised in the second construct is a constructive promoter or an inductive promoter, preferably psbA2 promoter, rbc promoter, petE promoter, cmp promoter, sbt promoter or trc promoter. More preferably the promoter as comprised in the second construct is rbc promoter or petE promoter.

Preferably the fatty acyl-CoA reducase gene is for example: far gene from Simmondsia chinensis (for example described in WO2011086189, the content of which is incorporated herein by reference); at3g11980 gene from Arabidopsis thaliana (for example described in WO2011086189, the content of which is incorporated herein by reference); far1 gene from mouse (see for example NCBI ID: BC007178); far1 gene with optimized codon from mouse; far2 gene from mouse (see for example NCBI ID: BC055759); or at3g56700 gene from Arabidopsis thaliana (see for example NCBI ID: NC_{—}003074.8). Other suitable fatty acyl-CoA reductase genes include Francci3_{—}2276 from Frankia sp. CcI3 (see for example NC_{—}007777); KRH_{—}18580 from Kocuria rhizophila DC2201 (see for example NC_{—}010617); A20C1_{—}04336 from Actinobacterium PHSC20C1 (see for example NZ_AAOB01000003); HCH_{—}05075 from Hahella chejuensis KCTC 2396 (see for example NC_{—}007645); Maqu_{—}2220 from Marinobacter aquaeolei VT8 (see for example NC_{—}008740); and RED65_{—}09889 from Oceanobacter sp. RED65 (see for example NZ_AAQH01000001).

Preferably, the second construct can further comprise a marker gene for screening the transformants of cyanobacteria, preferably kanamycin resistance gene, erythromycin resistance gene and spectinomycin resistance gene. Preferably the marker gene of the second construct is different from the marker gene of the first construct.
In a fifth aspect the present invention provides a kit, comprising two vectors, wherein the first vector comprises the first construct as defined above, and the second vector comprises the second construct as defined above.

Again, examples of vectors include but are not limited to cloning vectors and expression vectors. In a preferred embodiment, the vector is for example a plasmid, phage or coemid.

In a sixth aspect the present invention provides a cyanobacterium, which comprises the first construct as defined above and/or the first vector as defined above, and comprises the second construct as defined above and/or the second vector as defined above. Preferably this cyanobacterium is a cyanobacterium capable of producing fatty alcohols. More preferably this cyanobacterium is the cyanobacterium GQ5 as deposited at the China General Microbiological Culture Collection Center (CGMCC), having its address at the Institute of Microbiology, Chinese Academy of Sciences, No. 1 West Beichen Road, Chaoyang District, Beijing 100 101, China, under Accession Number of CGMCC 4890 on May 20, 2011.

In a seventh aspect the present invention provides a method for increasing the yield of fatty alcohols in one or more cyanobacteria capable of producing fatty alcohols, comprising introducing any of the above constructs and/or any of the above vectors into such one or more cyanobacteria.

Preferably, the cyanobacteria capable of producing fatty alcohols are those that are modified by genetic engineering to express fatty acyl-CoA reducase and thus can produce fatty alcohols. For example, the cyanobacteria capable of producing fatty alcohols can be obtained by introducing a gene encoding fatty acyl-CoA reducase into cyanobacteria, or integrating the gene into the genome of cyanobacteria. Suitable cyanobacteria include but are not limited to Synechocystis sp. Syn-XT14, Syn-XT34 and Syn-XT51 (such as described in WO2011086189, the content of which is incorporated herein by reference). Preferably the first construct is integrated into the genome of the cyanobacteria.

In an eight aspect the present invention provides a method for producing a fatty alcohol in one or more cyanobacteria, the method comprising:

1) introducing the first construct as defined above and/or the first vector as defined above, as well as the second construct as defined above and/or the second vector as defined above, into a cyanobacterium; and
2) culturing the cyanobacterium obtained in step 1), and obtaining fatty alcohols from the culture.
Preferably the cyanobacterium is Synechocystis sp. PCC6803.

Preferably, the first construct and/or the second construct are integrated into the genome of the cyanobacterium. Most preferably the cyanobacterium obtained in step 1) is cyanobacterium GQ5 as deposited in China General Microbiological Culture Collection Center (CGMCC) under Accession Number of CGMCC 4890 on May 20, 2011.

In another aspect, the embodiments of the present invention relate to a use of the first construct or the first vector as defined above for increasing the yield of fatty alcohols in cyanobacteria capable of producing fatty alcohols.

In another aspect, the embodiments of the present invention relate to the use of the kit as defined above for preparing a cyanobacterium capable of producing fatty alcohols.

In the present invention, the fatty alcohols are preferably fatty alcohols having a carbon chain length of at least 12 carbon atoms (for example, at least 13 carbon atoms, at least 14 carbon atoms, at least 15 carbon atoms, or at least 16 carbon atoms). More preferably they are fatty alcohols having a carbon chain length in the range from equal to or more than 12 carbon atoms to equal to or less than 20 carbon atoms. Most preferably the fatty alcohols are 1-hexadecanol and/or 1-octadecanol.

In a preferred embodiment these fatty alcohols can be converted into alkanes having a carbon chain length of at least 12 carbon atoms, more preferably alkanes having a carbon chain length in the range from equal to or more than 12 carbon atoms to equal to or less than 20 carbon atoms.

### EXAMPLES

The present invention is illustrated by the following examples. The examples are used only for the purpose of illustrating the present invention and are not intended to limit the protection scope of the present invention.

### Example 1

### Construction of the Vector for the Expression of Fatty Acyl-CoA Synthetase

In order to increase the expression level of fatty acyl-CoA synthetase in cyanobacteria, the plasmid pGQ7 carrying and expressing slr1609 gene was constructed as follows:

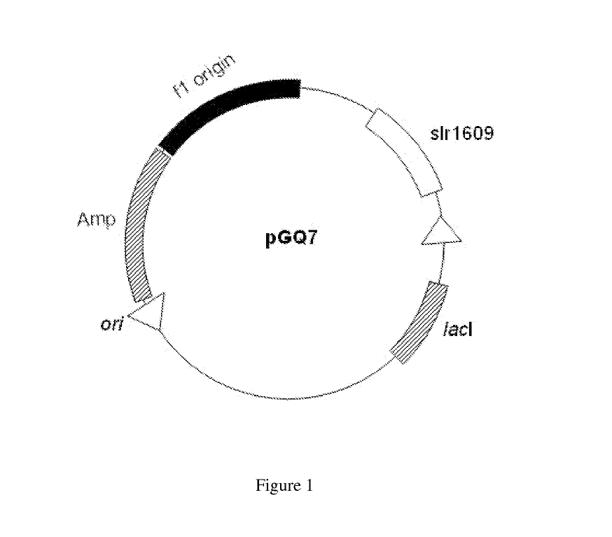

Polymerase chain reaction (PCR) amplification was performed using 1609NdeI (SEQ ID NO: 8, 5′-TAC ATA TGG ACA GTG GCC ATG GCG CTC AAT-3′) and 1609R (SEQ ID NO: 9, 5′-CCC TCG AGA AAC ATT TCG TCA ATT AAA TGT T-3′) as primers and using the genome DNA of Synechocystis sp. PCC6803 as template. The product of the PCR amplification was cloned into a pMD18-T vector (Takara, Catalog No.: D101A) according to the instructions of the manufacturer to obtain a plasmid pGQ3. After verification by sequencing, the plasmid pGQ3 was digested by using NdeI (Takara, Catalog No.: D1161A) and XhoI (Takara, Catalog No.: D1094A), and a DNA fragment of about 2.1 kb was recovered. In addition, the plasmid pET21b (Novagen) was digested by using NdeI (Takara, Catalog No.: D1161A) and XhoI (Takara, Catalog No.: D1094A), and the resulting DNA fragment was recovered. The two DNA fragments as obtained above were ligated by a ligase, resulting in the plasmid pGQ7 carrying slr1609 gene. The basic structure of the plasmid pGQ7, which comprised slr1609 gene (SEQ ID NO: 1), is shown in FIG. 1.

### Example 2

### Detection of the Activity of Fatty Acyl-CoA Synthetase

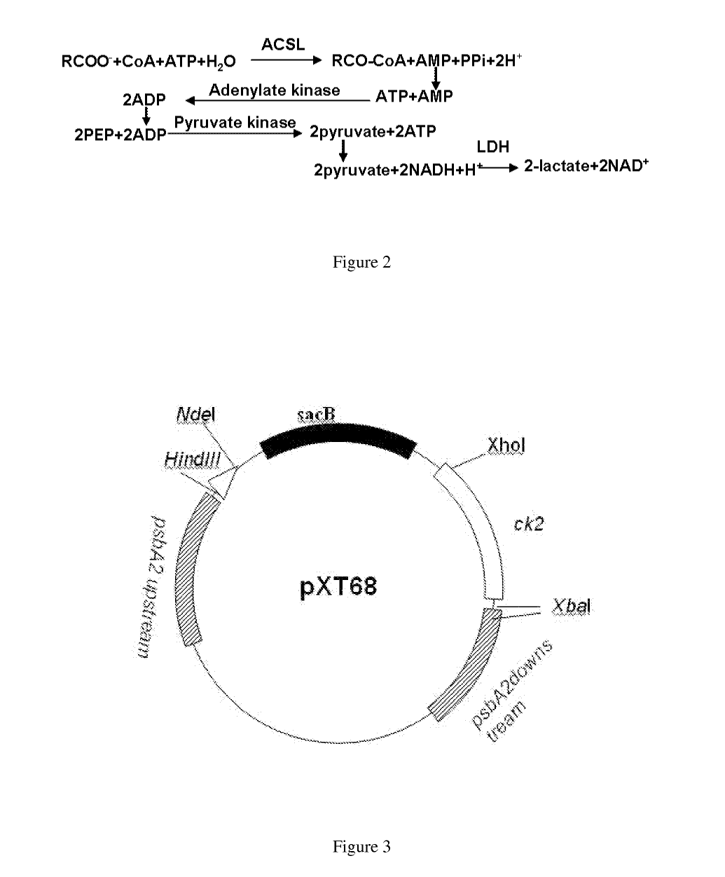

In order to determine whether the plasmid pGQ7 was capable of expressing functional fatty acyl-CoA synthetase, the activity of protein expressed by the plasmid pGQ7 was measured based on the coupling reactions as illustrated in FIG. 2. The specific reaction system was as follows: Tris-HCl (tris(hydroxymethyl)aminomethane-hydroxychloride) (pH7.4) 0.1 mM, dithiothreitol 5 mM, TritonX-100 1.6 mM, ATP 7.5 mM, magnesium chloride 10 mM, oleic acid 0.25 mM, coenzyme A (CoA) 1 mM, potassium phosphoenolpyruvate (PEPK) 0.2 mM, Nicotinamide adenine dinucleotide phosphate (NADH) 0.15 mM, adenylate kinase 11U, pyruvate kinase 9U, lactate dehydrogenase (LDH) 9U, the purified protein as expressed by plasmid pGQ7 (ACSL) 1.8 mM. Finally, the enzymatic activity was determined by measuring the optical absorption of NADH at 340 nm. The results showed that the protein as expressed by the plasmid pGQ7 had fatty acyl-CoA synthetase activity, and the k_{cat} value (the amount (mole) of substrate converted per mole of enzyme per minute), as measured by using oleic acid as substrate, was 3.0±0.3/min, the Kₘ value (Michaelis constant, i.e., the substrate concentration at which the reaction rate reaches the half of the maximum reaction rate) was 1.10±0.06 mM.

### Example 3

### Construction of the Vectors for Gene Knock-in and Gene Knock-Out

In order to verify the function of fatty acyl-CoA synthetase in the production of fatty alcohols in cyanobacteria, and to verify that the increase of yield of fatty alcohols in cyanobacteria can be achieved by increasing the expression of the enzyme, the vector pGQ49 for integrating the fatty acyl-CoA synthetase gene (slr1609 gene) driven by psbA2 promoter into the genome of cyanobacteria and the vector pGQ17 for knocking out the endogenous fatty acyl-CoA synthetase (slr1609 gene) in cyanobacteria were constructed as follows.

1. Construction of the Vector pXT68

PCR amplification was performed by using the genome DNA of Synechocystis sp. PCC6803 as template, with Pd1-2-f (SEQ ID NO: 14, 5′-CAC ATA GAT CTG CCA GTT GAG GT-3′) and Pd1-2-r (SEQ ID NO: 15, 5′-GGG CAT ATG GTT ATA ATT CCT TAT GTA TTT G-3′) as primers. The obtained PCR product was then cloned into a pMD18-T vector (Takara, Catalog No.: D101A) according to the instructions of the manufacturer, obtaining the plasmid pXT25. After verification by sequencing, the plasmid pXT25 was digested by PstI (Takara, Catalog No.: D1073A), and then end-filled by T4 DNA polymerase (Fermentas, Catalog No.: EP0061), and the resulted fragment of 4 kb was recovered. In addition, the plasmid pRL271 (SEQ ID NO: 18) was digested by EcoRV (Takara Catalog No.: D1040A) and XbaI (Takara Catalog No.: D1093A), and then end-filled by T4 DNA polymerase, and the resulted fragment of 3 kb was recovered (the fragment containing resistance gene). Then, the two fragments as obtained above were ligated by a ligase so as to obtain the plasmid pXT62.

PCR amplification was performed by using the genome DNA of Synechocystis sp. PCC6803 as template, with pD1-2d-1 (SEQ ID NO: 16, 5′-TTC CTT GGT GTA ATG CCA ACT G-3′) and pD1-2d-2 (SEQ ID NO: 17, 5′-TCC ACA CTG GGA AGT TTG CC-3′) as primers. The obtained PCR product was then cloned into pMD18-T vector (Takara, Catalog No.: D101A). Then, the resulted vector was digested by NdeI and SalI (Takara, Catalog No.: D1161A and D1080A), and then end-filled by T4 DNA polymerase. The final DNA fragment was recovered, and the vector pXT59 was obtained by self-linking the fragment.

The vector pXT62 was digested by XbaI and SphI (Takara, Catalog No.: D1093A and D1180), and then end-filled by T4 DNA polymerase, and the resulted fragment of 4.5 kb was recovered. The vector pXT59 was digested by XbaI, and then end-filled by T4 DNA polymerase, and the resulted fragment of 3.2 kb was recovered. The two fragments as obtained above were linked by using a ligase so as to obtain the plasmid pXT68. The basic structure of the plasmid pXT68 was shown in FIG. 3, which comprised the upstream fragment (SEQ ID NO: 6, comprising psbA2 promoter) and downstream fragment (SEQ ID NO: 7) of psbA2 gene as well as kanamycin resistance gene ck2 (SEQ ID NO: 4).

2. Construction of the Plasmid pGQ49

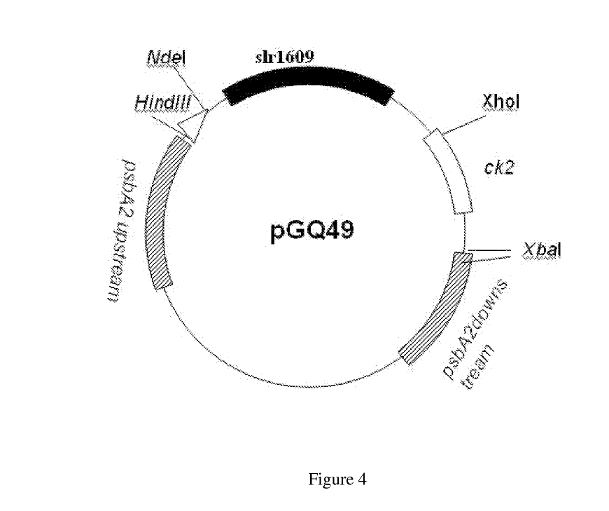

NdeI (Takara, Catalog No.: D1161A) and XhoI (Takara, Catalog No.: D1094A) were used to digest the plasmid pGQ7, and the resulted slr1609 gene fragment was recovered. Then the slr1609 gene fragment was inserted into the plasmid pXT68 that had been digested by NdeI and XhoI as well, so as to obtain the plasmid pGQ49. The basic structure of plasmid pGQ49 was shown in FIG. 4, which comprised the upstream fragment (SEQ ID NO: 6, comprising psbA2 promoter) of psbA2 gene, slr1609 gene (SEQ ID NO: 1), kanamycin resistance gene ck2 (SEQ ID NO: 4) and the downstream fragment (SEQ ID NO: 7) of psbA2 gene, and used for integrating the fatty acyl-CoA synthetase gene (slr1609 gene) driven by psbA2 promoter into the genome of cyanobacteria.

3. Construction of Plasmid pGQ17

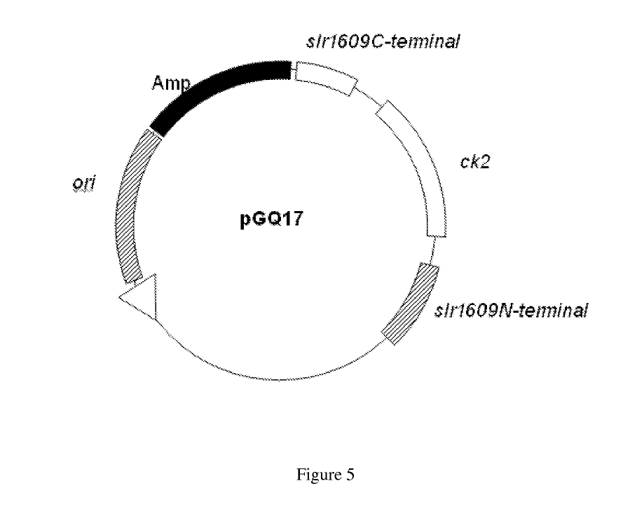

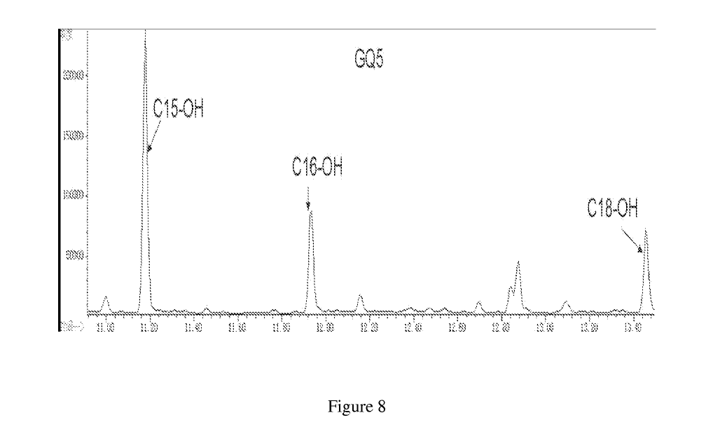

PCR amplification was performed by using the genome DNA of Synechocystis sp. PCC6803 as template, with 1609 kuF (SEQ ID NO: 10, 5′-TTT AAA TGG TGA TGA ACA CTG GGG A-3′) and 1609 kuR (SEQ ID NO: 11, 5′-GGG ATG ACT ATG GCG ATC GTT GAG-3′) as primers, and with 1609 kdF (SEQ ID NO: 12, 5′-TGT TTA CGC AGT GCC TAC ATT GA-3′) and 1609 kdR (SEQ ID NO: 13, 5′-CCC ATA GGC CTT AGA TCG TGT TT-3′) as primers, respectively. The obtained PCR products were cloned into pMD18-T vector (Takara, Catalog No.: D101A) respectively, to obtain the plasmids pGQ12 and pGQ13. The plasmid pRL446 (SEQ ID NO: 4) was digested by BamHI (Takara, Catalog No.: D1010A), and the obtained DNA fragment was then cloned into the vector pGQ12 that had been subjected to the same digestion as pRL446, so as to obtain the vector pGQ14. The vector pGQ14 was digested by DraI (Takara, Catalog No.: D1037A) and EcoRI (Takara, Catalog No.: D1040A), and the resulted DNA fragment of 1.6 kb comprising the upstream fragment of slr1609 gene and the ck2 gene was recovered. The DNA fragment was end filled with T4 DNA polymerase (Fermentas, Catalog No.: EP0061), then cloned into the vector pGQ13 that had been digested with SmaI (Takara, Catalog No.: D1085A), so as to obtain the plasmid pGQ17. The basic structure of the plasmid pGQ17 was shown in FIG. 5 which comprised the upstream fragment (SEQ ID NO: 2) of slr1609 gene, kanamycin resistance gene ck2 (SEQ ID NO: 4) and the downstream fragment of slr1609 gene (SEQ ID NO: 3), used for knocking out the endogenous fatty acyl-CoA synthetase gene (slr1609 gene) in cyanobacteria.

### Example 4

### Transformation of Cyanobacteria and Screening of Transformants

The transformation of cyanobacteria and the screening of transformants were performed as follows.

1. 10 mL of cyanobacteria cells in logarithmic growth phase (OD₇₃₀ of about 0.5-1.0) was taken, and centrifuged to collect the cell pellet; the cell pellet was washed twice with fresh BG11 medium, and then re-suspended in 1 mL BG11 medium (BG11 medium consisting of 1.5 g L⁻¹ NaNO₃, 40 mg L⁻¹ K₂HPO₄.3H₂O, 36 mg L⁻¹ CaCl₂.2H₂O, 6 mg L⁻¹ citric acid, 6 mg L⁻¹ ferric ammonium citrate, 1 mg L⁻¹ EDTA disodium salt, 20 mg L⁻¹ NaCO₃, 2.9 mg L⁻¹ H₃BO₃, 1.8 mg L⁻¹ MnCl₂.4H₂O, 0.22 mg L⁻¹ ZnSO₄.7H₂O, 0.39 mg L⁻¹ NaMoO₄.2H₂O, 0.079 mg L⁻¹ CuSO₄.5H₂O and 0.01 mg L⁻¹ CoCl₂.6H₂O).

2. 0.2 mL of cell suspension was placed in a new EP tube; 2-3 μg of the expression plasmid as listed in Table 1 was added into the tube, mixed well and incubated at 30° C. under an illumination condition of 30 μE m⁻² s⁻¹ for 5 hours.

3. The mixture of cyanobacteria cells and DNA was plated onto a nitrocellulose membrane on a BG11 plate (without antibiotics) and cultivated at 30° C. under an illumination condition of 30 μE m⁻² s⁻¹ for 24 hours. Then, the nitrocellulose membrane was transferred to a BG11 plate containing a antibiotic corresponding to the desired strain (see Table 1), and further incubated at 30° C. under a condition of 30 μE m⁻² s⁻¹.

4. After culturing for about 5-7 days, the transformants were picked out from the plate, and used to streak a fresh BG11 plate (containing a corresponding antibiotic). After the cells were enriched, they are inoculated into a liquid BG11 medium (containing a corresponding antibiotic) for further cultivation.

5. After the transformed cyanobacteria cells were transferred twice or thrice in liquid BG11 medium (containing a corresponding antibiotic) and the introduction of the desired construct was confirmed by genome sequencing, the transformed cells were used for measuring the yield of fatty alcohols.

TABLE 1
The cyanobacteria strains used and their antibiotic resistance

| Strain | Source/way to obtain | Resistance |
|---|---|---|
| PCC6803 | Wild type | — |
| Syn-XT14 | Tan, et al, 2011; see WO2011086189 herein | Spectinomycin |
| | incorporated by reference | (10 μg mL⁻¹) |
| | | resistance |
| GQ5 | Obtained by transforming Syn-XT14 with | Spectinomycin |
| | plasmid pGQ49 | (10 μg mL⁻¹) + |
| | | kanamycin |
| | | (10 μg mL⁻¹) |
| | | resistance |
| GQ6 | Obtained by transforming Syn-XT14 with | Spectinomycin |
| | plasmid pGQ17 | (10 μg mL⁻¹) + |
| | | kanamycin |
| | | (10 μg mL⁻¹) |
| | | resistance |

Genotype of cyanobacteria strains:
PCC6803: wild-type Synechocystis sp. PCC6803, glucose-tolerant.
Syn-XT14: slr0168::Omega Prbc far (jojoba) Trbc: comprising FAR gene that was driven by rbc promoter and originated from jojoba (integrated at the site of slr0168 gene), spectinomycin resistant.
GQ5: slr0168::omega Prbc far (jojoba), psbA2::CK2 PpsbA2 slr1609: comprising FAR gene (integrated at the site of slr0168 gene) that was driven by rbc promoter and originated from jojoba, spectinomycin resistant, and comprising slr1609 gene (integrated at the site of psbA2 gene) driven by psbA2 promoter, kanamycin resistant.
GQ6: slr0168::omega Prbc far (jojoba), slr160::CK2: comprising FAR gene (integrated at the site of slr0168 gene) that was driven by rbc promoter and originated from jojoba, spectinomycin resistant, with endogenous slr1609 gene being knocked out, kanamycin resistant.

### Example 5

### Yield of Fatty Alcohols of Cyanobacteria Modified by Genetic Engineering

1. Experimental Steps:

(1) Culturing manner: shaking culture. To a normal 500 mL conical flask with 300 mL liquid BG11 medium (containing an antibiotic corresponding to the desired cyanobacteria strain; as for the wild type cyanobacteria strain, containing no antibiotic), the initial inoculation concentration was OD₇₃₀=0.05, and the culturing was performed at 30° C. and illumination condition of 30 μE m⁻² s⁻¹ with air for 7-8 days;

(2) 200 mL of culture was taken, and cyanobacteria cells were collected by centrifugation, re-suspended with 10 mL TE (Tris(hydroxymethyl)aminomethane-Ethylenediaminetetraacetic acid)

pH8.0 buffer solution, and then broken using ultrasonic waves;

(3) To the cell lysates, 30 μg pentadecanol as internal standard was added, the solution of chloroform:methanol (v/v 2:1) was added isovolumetrically, mixed homogeneously, and left at room temperature for 0.5 hours;

(4) The organic phase was recovered by low speed centrifugation (3,000 g) for 5 min, and dried with nitrogen blast at 55° C.;

(5) The deposit was dissolved by adding 1 mL of n-hexane, and filtered with 0.22 μm filtration membrane, and Gas Chromatography-Mass Spectrometry (GC-MS) analysis was performed with Agilent 7890A-5975C system and Agilent HP-INNOWax (30 m×250 μm×0.25 μm) according to the manufacturer's specification to determine the contents of various fatty alcohols. The conditions of analysis were as follows: carrier gas was helium gas; flow rate was 1 mL/min; sample introduction inlet temperature was 250° C.; the temperature programming of column box was as follows: 100° C., 1 min; then elevated in a rate of 5° C./min to 200° C.; then elevated in a rate of 25° C./min to 240° C.; held for 15 min.

2. Experimental Results:

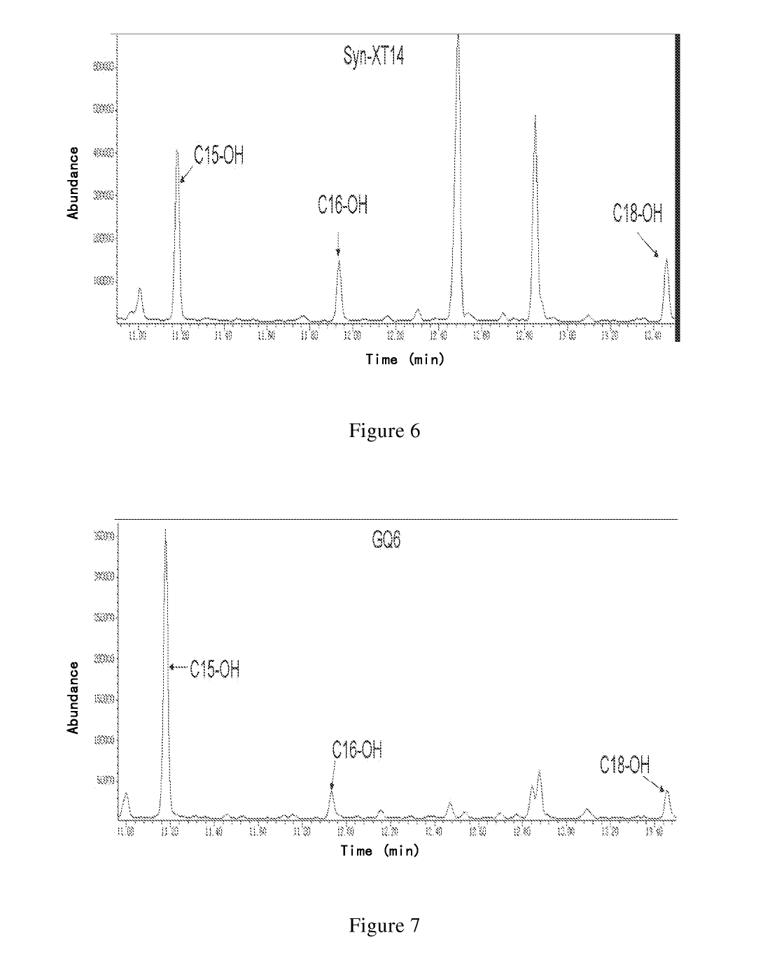

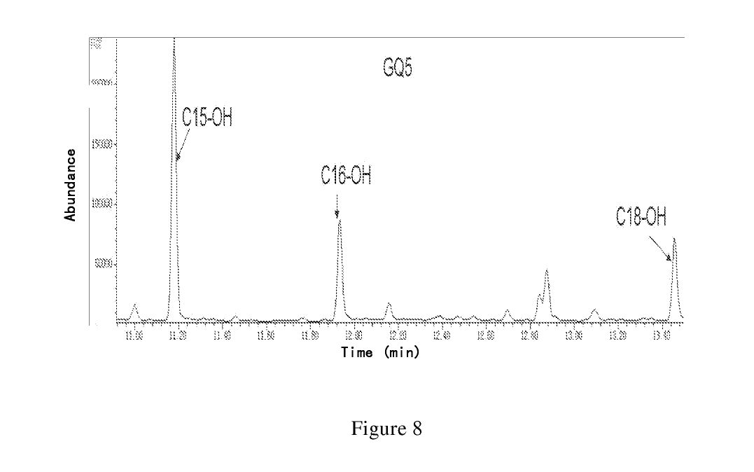

We detected hexadecanol and octodecanol in three strains of genetic engineered cyanobacteria, Syn-XT14, GQ5 and GQ6, respectively, but did not detect the production of fatty alcohols in the wild type cyanobacteria PCC6803 (see: FIGS. 6-8). FIGS. 6-8 showed the production of fatty alcohols in cells of Synechocystis sp. Syn-XT14, GQ6 and GQ5 respectively, as measured by using gas chromatography coupled with mass spectrometry (GC-MS).

By referring to the internal standard (pentadecanol), the total amount of fatty alcohols in cells under normal shaking culturing conditions can be calculated, and the results are shown in Table 2. The results show that free fatty acids are catalyzed by fatty acyl-CoA synthetase coded by slr1609 gene to form fatty acyl-CoAs, the latter are further used as substrates of fatty acyl-CoA reducase and converted into fatty alcohols. Table 2 also shows that, as compared to the yield of Syn-XT14, in GQ5, the hexadecanol yield is elevated by about 53%, the octadecanol yield is elevated by about 59%, and the total yield of fatty alcohols is elevated by about 57%. In addition, as compared to the yield of Syn-XT14, in GQ6, the hexadecanol yield, the octadecanol yield and total yield of fatty alcohols significantly decrease. These results show that the increase of slr1609 gene expression level results in a corresponding increase of yield of fatty alcohols in cyanobacteria, and the decrease of expression level of said gene results in a corresponding decrease of yield of fatty alcohols in cyanobacteria.

Hence, the present invention sufficiently confirms the important effects of fatty acyl-CoA synthetase gene on the production of fatty alcohols in cyanobacteria, and confirms that the yield of fatty alcohols in cyanobacteria can be elevated by increasing the expression level of fatty acyl-CoA synthetase, which provides beneficial conditions for production of fatty alcohols as biofuel in large scale using cyanobacteria. If so desired the fatty alcohols that are produced can conveniently be converted into alkanes, for example by means of hydrogenation. These alkanes can be useful as component in a biofuel or biochemical.

TABLE 2
Yields of fatty alcohols of the used cyanobacteria strains (unit: μg/L/OD)

| Strain | Hexadecanol | Octadecanol | Total yield |
|---|---|---|---|
| PCC6803 | N.D | N.D | N.D |
| Syn-XT14 | 6.07 ± 1.06 | 6.52 ± 1.00 | 12.6 ± 2.05 |
| GQ5 | 9.32 ± 0.20 | 10.4 ± 2.10 | 19.8 ± 2.31 |
| GQ6 | 2.07 ± 0.27 | 2.89 ± 0.38 | 4.97 ± 0.11 |

Notation:
N.D = Not detectable

Although the specific embodiments of the present invention have been described in details, those skilled in the art would understand that, according to the teachings disclosed in the specification, those details can be modified and changed without departing from the sprit or scope of the present invention as generally described. The scope of the present invention is given by the appended claims and any equivalents thereof.

Deposition Information of the Samples of Biological Materials

The cyanobacteria strains Syn-XT14, Syn-XT34, Syn-XT51, and GQ5 as mentioned in the present invention are all deposited in China General Microbiological Culture Collection Center (CGMCC) (having its address at the Institute of Microbiology, Chinese Academy of Sciences, No. 1 West Beichen Road, Chaoyang District, Beijing 100 101, China), and their deposition dates and accession numbers are shown in Table 3.

TABLE 3
Cyanobacteria strains as involved and their deposition information

| Strains | Accession No. | Deposition date |
|---|---|---|
| Cyanobacteria Syn-XT14 | CGMCC 3894 | Jun. 10, 2010 |
| Cyanobacteria Syn-XT34 | CGMCC 3895 | Jun. 10, 1010 |
| Cyanobacteria Syn-XT51 | CGMCC 3896 | Jun. 10, 2010 |
| Cyanobacteria GQ5 | CGMCC 3948 | May 20, 2011 |

What is claimed is:

1. A construct comprising a gene operably linked to a promoter having activity in cyanobacteria, wherein said gene is selected from the group consisting of:
1) fatty acyl-CoA synthetase genes;
2) genes of which the nucleotide sequences have at least 80% identity to the sequences of the genes listed in 1), and which encode a protein having fatty acyl-CoA synthetase activity; and
3) genes of which the nucleotide sequences are capable of hybridizing with the sequences of the genes listed in 1) under stringent hybridizing conditions, and which encode a protein having fatty acyl-CoA synthetase activity.

2. The construct of claim 1 wherein the construct further comprises a marker gene for screening the transformants of cyanobacteria.

3. The construct of claim 2 wherein the marker gene is kanamycin resistance gene, erythromycin resistance gene or spectinomycin resistance gene.

4. The construct of claim 2 wherein the marker gene is located upstream or downstream of the promoter having activity in cyanobacteria.

5. The construct of claim 1 wherein there exists an upstream fragment and a downstream fragment of the psbA2 gene respectively at the two ends of the construct.

6. A vector comprising the construct of claim 1.

7. A vector comprising the construct of claim 2.

8. A cyanobacterium comprising the construct of claim 1.

9. A cyanobacterium comprising the construct of claim 2.

10. A cyanobacterium comprising the vector of claim 6.

11. A kit comprising two constructs, wherein the first construct is a construct of claim 1, and the second construct comprises a gene operably linked to a promoter having activity in cyanobacteria, said gene is selected from the group consisting of:
1) fatty acyl-CoA reductase genes;
2) genes of which the nucleotide sequences have at least 80% identity to the sequences of the genes listed in 1), and which encode a protein having fatty acyl-CoA reducase activity; and
3) genes of which the nucleotide sequences are capable of hybridizing with the sequences of the genes listed in 1) under stringent hybridizing conditions and which encode a protein having fatty acyl-CoA reducase activity.

12. A kit comprising two constructs, wherein the first construct is a construct of claim 2 and the second construct comprises a gene operably linked to a promoter having activity in cyanobacteria, said gene is selected from the group consisting of:
1) fatty acyl-CoA reductase genes;
2) genes of which the nucleotide sequences have at least 80% identity to the sequences of the genes listed in 1), and which encode a protein having fatty acyl-CoA reducase activity; and
3) genes of which the nucleotide sequences are capable of hybridizing with the sequences of the genes listed in 1) under stringent hybridizing conditions and which encode a protein having fatty acyl-CoA reducase activity.

13. A kit comprising two vectors, wherein the first vector comprises a vector of claim 6, and the second vector comprises a vector comprising a second construct which comprises a gene operably linked to a promoter having activity in cyanobacteria, said gene is selected from the group consisting of:
1) fatty acyl-CoA reductase genes;
2) genes of which the nucleotide sequences have at least 80% identity to the sequences of the genes listed in 1), and which encode a protein having fatty acyl-CoA reducase activity; and
3) genes of which the nucleotide sequences are capable of hybridizing with the sequences of the genes listed in 1) under stringent hybridizing conditions and which encode a protein having fatty acyl-CoA reducase activity.

14. A cyanobacterium comprising a first construct, wherein the first construct is a construct of claim 1, and a second construct comprising a gene operably linked to a promoter having activity in cyanobacteria, said gene is selected from the group consisting of:
1) fatty acyl-CoA reductase genes;
2) genes of which the nucleotide sequences have at least 80% identity to the sequences of the genes listed in 1), and which encode a protein having fatty acyl-CoA reducase activity; and
3) genes of which the nucleotide sequences are capable of hybridizing with the sequences of the genes listed in 1) under stringent hybridizing conditions and which encode a protein having fatty acyl-CoA reducase activity.

15. A method for increasing the yield of fatty alcohols in one or more cyanobacteria capable of producing fatty alcohols, comprising introducing the construct of claim 1 into the one or more cyanobacteria.

16. A method for increasing the yield of fatty alcohols in one or more cyanobacteria capable of producing fatty alcohols, comprising introducing the construct of claim 2 into the one or more cyanobacteria.

17. A method for increasing the yield of fatty alcohols in one or more cyanobacteria capable of producing fatty alcohols, comprising introducing the vector of claim 6 into the one or more cyanobacteria.

18. A method for producing fatty alcohol in cyanobacteria comprising:
1) introducing a first construct, wherein said first construct is a construct of claim 1, as well as a second construct wherein said second construct comprises a gene operably linked to a promoter having activity in cyanobacteria, said gene is selected from the group consisting of:
a) fatty acyl-CoA reductase genes;
b) genes of which the nucleotide sequences have at least 80% identity to the sequences of the genes listed in a), and which encode a protein having fatty acyl-CoA reducase activity; and
c) genes of which the nucleotide sequences are capable of hybridizing with the sequences of the genes listed in a) under stringent hybridizing conditions and which encode a protein having fatty acyl-CoA reducase activity into a cyanobacterium; and
2) culturing the cyanobacterium obtained in step 1), and
3) obtaining fatty alcohols from culture.

19. The method of claim 18 further comprising converting the obtained fatty alcohols to alkanes.

20. The method of claim 19 further comprising blending the alkanes with one or more additives into a fuel.
